SEQUENCE LISTING (11 sequences)

SEQ ID NO: 1 - DNA, 46 bp, synthetic construct
  source 1..46: mol_type = other DNA
gggagatgccactgatgagtccgtgaggacgaaacggtataccgtc

SEQ ID NO: 2 - DNA, 402 bp, synthetic construct
  source 1..402: mol_type = other DNA
aaatatatttgcaatcaaaagttatcaggcatgcacctggtagctagtctttaaaccaatagattgcatcggtttaaaaggcaagaccgtcaaattgcgggaaaggggtcaacagccgttcagtaccaagtctcaggggaaactttgagatggccttgcaaagggtatggtaataagctgacggacatggtcctaaccacgcagccaagtcctaagtcaacagatcttctgttgatatggatgcagttcacagactaaatgtcggtcggggaagatgtattcttctcataagatatagtcggacctctccttaatgggagctagcggatgaagtgatgcaacactggagccgctgggaactaatttgtatgcgaaagtatattgattagttttggagtactc

SEQ ID NO: 3-4 - skipped (2 entries)

SEQ ID NO: 5 - DNA, 17 bp, synthetic construct
  source 1..17: mol_type = other DNA
taagcggcaggcggaag

SEQ ID NO: 6 - DNA, 22 bp, synthetic construct
  source 1..22: mol_type = other DNA
tagttcgggtttcgtcagatgg

SEQ ID NO: 7 - DNA, 21 bp, synthetic construct
  source 1..21: mol_type = other DNA
tggcagtgtcttagctggttg

SEQ ID NO: 8 - DNA, 21 bp, synthetic construct
  source 1..21: mol_type = other DNA
gcgagcacagaattaatacga

SEQ ID NO: 9 - DNA, 49 bp, synthetic construct
  source 1..49: mol_type = other DNA
gcgagcacagaattaatacgactcactataggtttttttttttttttac

SEQ ID NO: 10 - DNA, 22 bp, synthetic construct
  source 1..22: mol_type = other DNA
tggcagtgtcttagctggttgt

SEQ ID NO: 11 - RNA, 23 nt, synthetic construct
  source 1..23: mol_type = other RNA
tggcagtgtctttagctggttgt

(19) United States
(12) Patent Application Publication
(10) Pub. No.: US 2026/0234618 A1
(43) Pub. Date: Aug. 13, 2026

(54) SELF-AMPLIFYING NON-CODING RNA FOR TARGETED GENE KNOCKDOWN

(71) Applicant: The Regents of the University of California, Oakland, CA (US)

(72) Inventors: William M. Gelbart, Los Angeles, CA (US); Charles M. Knobler, Los Angeles, CA (US); Feng Guo, Los Angeles, CA (US)

(73) Assignee: The Regents of the University of California, Oakland, CA (US)

(21) Appl. No.: 19/150,483

(22) PCT Filed: Feb. 2, 2024
(86) PCT No.: PCT/US24/14231

§ 371 Date: Jul. 23, 2025

Related U.S. Application Data
(60) Provisional application No. 63/482,857, filed on Feb. 2, 2023.

(51) Int. Cl. C12N 15/113; C12N 5/071; C12N 7/00; C12N 9/12; C12N 15/85; C12N 15/88

(52) U.S. Cl. CPC C12N 15/113; C12N 5/0686; C12N 7/00; C12N 9/127; C12N 15/85; C12N 15/88; C12N 2310/121; C12N 2310/1241; C12N 2310/141; C12N 2510/00; C12N 2770/30022; C12Y 207/07048

(57) ABSTRACT

We describe the construction of a self-replicating form of microRNA (miRNA) and demonstrate its amplification and biological activity in cultured cells. In typical embodiments, a miRNA sequence of interest is flanked by self-cleaving ribozymes and added to RNAl of the Nodamura virus, which is directly translated to generate an RNA-dependent-RNA-polymerase that replicates it strongly along with the doubly-self-cleaving ribozyme cassette containing the miRNA. In the specific case of miR-34a flanked by Hammerhead and Tetrahymena ribozymes, BHK-21 cells transfected with the replicon form of this cassette are shown to generate up to one million copies/cell of the miRNA. The biological activity of this amplified miR-34a is demonstrated by the essentially complete apoptosis of cultured prostate cancer cells transfected with the replicon. More generally, we provide this self-amplifying form of miRNA as a means for restoring homeostatic levels of miRNA in cancer cells, as well as for targeting specific gene expression.

### CROSS REFERENCE TO RELATED APPLICATIONS

This application claims the benefit under 35 U.S.C. Section 119(e) of co-pending and commonly-assigned U.S. Provisional Patent Application No. 63/482,857, filed Feb. 2, 2023, entitled “SELF-AMPLIFYING NON-CODING RNA FOR TARGETED GENE KNOCKDOWN”, which applications are incorporated by reference herein.

### TECHNICAL FIELD

The present invention relates to self-amplifying RNAs and methods for making and using them.

### BACKGROUND OF THE INVENTION

Since its discovery 30 years ago, microRNA (miR) gene regulation and RNA interference (RNAi) has burgeoned quickly into a dominant and thriving medical therapeutic industry, in part because this knowledge and technology allow for direct gene knockdown in a highly specific and personalized fashion.

However, conventional methods of introducing micro-RNAs and small interfering RNAs (siRNAs) into cells have involved transcription from a DNA plasmid or retroviral or DNA viral vector, thereby necessarily involving traffic in and out of the nucleus. Other methods involve direct transfection of these interfering RNAs into the cytoplasm, often complexed with cationic polymer or lipid. However these methods are limited in the number of copies that can be delivered.

For the reasons noted above, there is a need in the art for new self-amplifying RNA materials and methods for making and using them.

### SUMMARY OF THE INVENTION

As disclosed herein, we have discovered that it is possible to exploit replication strategies of the Nodaviridae family of viruses in order to design and develop a number of systems for making and using self-amplifying RNAs. As illustrative embodiments of the invention we constructed a self-replicating (“replicon”) form of microRNA (miRNA) and then demonstrated its amplification and biological activity in mammalian cells.

In embodiments of the invention, structural and functional elements in a polyribonucleotide are selected, organized and generated so that cargo RNA of interest is flanked by self-cleaving ribozymes so as to form a doubly-self-cleaving ribozyme cassette. In embodiments of the invention, the polyribonucleotide further comprises additional elements including a polyribonucleotide sequence encoding a RNA dependent RNA polymerase such as RNA1 of the Nodamura virus. Translation of this polyribonucleotide then generates a RNA-dependent RNA polymerase that replicates the polyribonucleotide along with the doubly-self-cleaving ribozyme cassette containing the cargo RNA. In a working embodiment of the invention that is discussed below, the polyribonucleotide cargo comprises a miR-34a RNA flanked by Hammerhead and Tetrahvmena ribozymes. Such self-amplifying forms of miRNA have a number of applications, including their use as a means for restoring homeostatic levels of miRNA in cancer cells, as well as for targeting specific gene expression. Illustrating this, mammalian cells transduced with a polyribonucleotide embodiment of the invention that comprises a miRNA-34a cargo sequence can generate up to one million copies/cell of this miRNA cargo. The biological activity of the amplified miR-34a polyribonucleotides generated in this embodiment of the invention were confirmed by observing the essentially complete apoptosis of cultured prostate cancer cells transfected with the polyribonucleotide comprising this miR-34a cargo.

Advantages of the invention disclosed herein include that a siRNA or microRNA can be introduced via an RNA molecule that is directly translated in the cytoplasm in order to produce a desired RNA therapeutic. In such embodiments, this microRNA molecule is amplified up to one-million-fold due to its being embedded in an RNA replicon under the control of a subgenomic promoter. In such embodiments, the nucleus is not involved at all in the generation of the desired RNA therapeutic. Further, with such embodiments, the polynucleotides of the invention are localized precisely at the desired sites—namely, the ribosomes—for gene knockdown by mRNA translation repression.

The invention disclosed herein has a number of embodiments. Embodiments of the invention include compositions of matter comprising a self-replicating polynucleotide. Typically in such compositions the polynucleotide comprises a constellation of elements including a polynucleotide sequence encoding a RNA dependent RNA polymerase: a T2A polynucleotide sequence encoding a self-cleaving peptide sequence; a polynucleotide sequence comprising a first (“left” (5′)-flanking) ribozyme: a polynucleotide sequence comprising a cargo (e.g., microRNA) polynucleotide; and a polynucleotide sequence comprising a second (“right” (3′)-flanking) ribozyme. Note that when we refer here and throughout to “self-cleaving peptide” sequence encoded by RNA polynucleotide sequence, we are—more precisely—describing a polynucleotide sequence that, when translated, results in a peptide bond not forming between two successive amino acids encoded by it. In such compositions, these elements are disposed in the polynucleotide such that when the polynucleotide is transduced into a mammalian cell the RNA-dependent-RNA-polymerase (RdRp) encoded by the polynucleotide is made, the polynucleotide is replicated, and the two self-cleaving flanking ribozymes cleave to release the cargo (e.g., microRNA) fragment. Optionally, the cargo polynucleotide comprises a MiRNA. In certain embodiments of the invention, the polynucleotide is disposed within a Nodamura (“Noda”) T2A vector.

Embodiments of the invention include methods of making the compositions disclosed herein. Typically such methods comprise forming a polynucleotide, wherein the polynucleotide is formed to include a constellation of elements such as a polynucleotide sequence encoding a RNA-dependent-RNA-polymerase, a T2A self-cleaving peptide sequence, a polynucleotide sequence comprising a first ribozyme, a polynucleotide sequence comprising a cargo polynucleotide, and a polynucleotide sequence comprising a second ribozyme. These methods include organizing the constellation of elements in the polynucleotide such that when the polynucleotide is transduced into a mammalian cell the RNA-dependent-RNA-polymerase encoded by the polynucleotide is made, the polynucleotide is replicated, and the first and second ribozymes cleave the polynucleotide such that a polynucleotide fragment consisting of the cargo polynucleotide is generated. A variety of different elements can be used to make such polynucleotides both in vitro and in vivo. For example, in certain embodiments of the invention, the polynucleotide sequence encoding a RNA-dependent-RNA-polymerase (RdRp) corresponds to the Nodaviridae RdRp, the polynucleotide sequence comprising the first ribozyme or the polynucleotide sequence comprising the second ribozyme comprise a Hammerhead ribozyme and a Tetrahymena ribozyme, and/or the polynucleotide sequence comprising a cargo polynucleotide consists of a miRNA. In typical embodiments, the elements in the polynucleotide are selected and organized such that when the polynucleotide is transduced into a mammalian BHK-21 cell, at least 5,000 polynucleotide fragments consisting of the cargo polynucleotide are generated.

Embodiments of the invention also include methods of delivering a cargo polynucleotide into a mammalian cell either in vitro or in vivo, the method comprising uptake by the mammalian cell (e.g. a cancer cell such as a pancreatic cancer cell or prostate cancer cell) with a self-replicating polynucleotide composition disclosed herein under conditions selected so that the cargo polynucleotide is transduced into the mammalian cell. A variety of conventional methodologies can be used to transduce the mammalian cell. Optionally, for example, in in vitro cell culture work, the cargo polynucleotide is transduced into the mammalian cell via a lipofection method. Typically in these methods, when the cargo polynucleotide is transduced into the mammalian cell, at least 2,000 polynucleotide fragments consisting of the cargo polynucleotide are generated. For in vivo delivery, the cargo polynucleotide is packaged in a virus-like particle (VLP) or enveloped virus-like particle (EVLP) as described in U.S. Pat. No. 9,605,031 and Provisional Patent Application No. Ser. No. 63/482,704, filed on Feb. 1, 2023, the contents of which are incorporated by reference.

Other objects, features and advantages of the present invention will become apparent to those skilled in the art from the following detailed description. It is to be understood, however, that the detailed description and specific examples, while indicating some embodiments of the present invention, are given by way of illustration and not limitation. Many changes and modifications within the scope of the present invention may be made without departing from the spirit thereof, and the invention includes all such modifications.

### BRIEF DESCRIPTION OF THE DRAWINGS

FIG. 1: Replication Scheme of the NodaT2A Replicon. Replication of the NodaT2A replicon-derived from RNA1 of the two-molecule Nodamura genome—begins with the 5′-capped viral mRNA (positive-sense strand) being translated by ribosomes starting at initiator codon AUG, generating a replicase protein (Protein A) that binds the 5′- and 3′-untranslated regions (UTRs) and replicates the molecule, producing an RNA molecule that is its reverse-complement (negative[−]-sense strand). The newly synthesized minus strand and the original positive-sense strand form a transient double-stranded intermediate to which protein A binds and transcribes new positive-sense RNA molecules, identical to the initial RNA molecule that was translated. Successive cycles of this process generate large numbers of the replicon.

FIG. 2: Design and Structure of the Core Catalytic Sequence: Self-Cleaving miRNA. The core catalytic sequence (CCS) is composed of three parts. The mature miRNA, whose 22-nt sequence is shown above, is flanked by two ribozymes. The 5′ end contains a hammerhead ribozyme (46 nt), designed to cleave directly upstream of the first nucleotide of the microRNA. The 3′ end of the sequence contains a modified group-I intron (402 nt) from Tetrahymena, which was chosen to ensure that the 3′ end of the miRNA is terminated with a hydroxyl group; the G shown with an arrow is the guanosine cofactor needed to activate the ribozyme. The 5′ hydroxyl resulting from the cleaving of the 5′ hammerhead ribozyme is subsequently phosphorylated by cellular kinases, resulting in a mature miRNA.

FIG. 3: Incorporation of the CCS into the NodaT2A replicon. Full-length NodaMiRNA RNA, with 5′ and 3′ UTRs and containing the CSS cassette, is replicated by Protein A to yield the negative-sense RNA that is catalytically inactive. This serves as a new template molecule for Protein A, from which new NodaMiRNA molecules are synthesized via transcription. The new NodaMiRNA RNAs, identical to the initial RNA molecule, cleave to generate 5′ and 3′ products along with the miRNA precursor molecules that become functional miRNA upon phosphorylation of the 5′ hydroxyl.

FIG. 4: 20% Urea PAGE gel showing miRNA produced from cleavage of in vitro transcribed CCS. 20% denaturing Urea-PAGE image showing the gradual accumulation, co-transcriptionally, of miR-34a. The far-right lane corresponds to the microRNA ladder with lengths of 17, 21, and 25 nt. During transcription, aliquots of transcription mix were taken at 5, 20, 40, 60, 120, and 180 minutes post-reaction start. A strong band at 22 nt represents the cleaved miRNA molecule. Note at earlier times (20 minutes) partially cleaved bands appear above the miRNA band of interest.

FIG. 5: In Vitro Cleavage of NodaMiRNA. Replicon molecules were generated via in vitro transcription with an Invitrogen MMESSAGE MMACHINE T7 transcription kit. Aliquots were taken at six time points (t=0, 15, 30, 45, 60, 120 minutes, shown left to right above) and run in lanes 1-6 from left to right. A ladder is shown in the far-right lane, labels in knt. The faint bands at 3.5 knt represent full-length NodaMiRNA (*) molecules, while the stronger bands at 3 knt represent the 5′ cleavage product (**). The 3′ cleavage product is visible at approximately 600 nt (***) starting at 15 minutes post-transcriptional start and becomes intense at 1 hour post-transcriptional start, when approximately 75% of the molecules produced have cleaved. Band density near the well represents the DNA template, and smearing is representative of incomplete cleavage.

FIG. 6: Time Course Gel of Purified Cellular RNAs from BHK-21 Cells Transfected with 2 μg of NodaM\iRNA. Total cellular RNAs from approximately 1 million cells were purified using a Qiagen RNeasy column, at five time-points post-transfection (8, 18, 24, 48, and 72 hours, lanes 2-6). 1 μg total RNA was denatured, loaded onto a 1.2% agarose TAE gel, and run for 2 hours. An ssRNA ladder (2 g NEB ssRNA ladder, right, labels in knt), 500 ng of in vitro transcribed (IVT) NodaMiRNA only containing the 5′ and 3′ cleavage products (adjacent to 72-hour time point), as well as RNA from untransfected BHK-21 cells (far left, U.T.), were also run as internal standards. Strong amplification of full-length NodaMiRNA RNAs (*) is visible in transfected samples beginning 18 hours post-transfection, and the 5′ and 3′ cleavage products as well (**, ***). Subgenomic RNAs are visible at approximately 1.1 knt (o). The band that runs at around 3.5 knt represents both full-length NodaMiRNA RNA as well as the minus-strand intermediate, which is the same length but does not cleave. The very strong bands just above (*) and above (o) are the 28S and 18S ribosomal RNAs.

FIG. 7: Positive-Sense Noda Molecule Time Course: Total cellular RNAs from BHK-21 cells transfected with 2 μg NodaMiRNA were harvested at several points post-transfection using an RNeasy column. One μg of purified RNAs was reverse transcribed using NEB MMULV reverse transcriptase with a reverse primer targeting the T2A region of the RNA molecule. The reverse transcription reactions were diluted to a final concentration of 100 pg/μL, and one μL of the resulting solution was used for qPCR analysis. Assays were performed in triplicate over the course of three separate transfection experiments; horizontal lines represent the average of the independent experiments. Error bars (vertical lines) correspond to the standard deviation.

FIG. 8: A MicroRNA Time Course: The small-molecule enriched portions of total cellular RNAs were harvested from BHK-21 cells at the indicated time points post-transfection with 2 μg NodaMiRNA RNA. 1 μg of these RNAs was first polyadenylated using NEB polyA-polymerase. The polyadenylated microRNAs were then reverse transcribed using an adapter molecule that hybridized to the 3′ ends of the microRNA, as well as the polyA tail. The reverse transcription reaction was performed using NEB MMULV reverse transcriptase. This reverse transcription reaction mix was diluted to a concentration of 100 pg/μL, and 1 μL of the resulting solution was analyzed in triplicate. Assays were performed over the course of three separate transfection experiments and then averaged (black horizontal bars). Error bars reported in standard deviation.

FIG. 9: NodaMiRNA constructs induce apoptosis in PC3 cells cultured in vitro. PC3 cells were transfected with 1.5 μg of NodaMiRNA or NodaEYFP RNA, or were left untransfected (naïve.) To ensure replication, these cells were incubated at 30° C. for 24 hours, then transferred to 37° C. At the indicated time post-transfection cells were collected, stained with trypan blue, and counted with a hemocytometer. The ratio (≤1:10) of dead to live cells for the naïve cells remained constant, as did the ratio (≤1:2% for cells transfected with Nodamura EYFP replicon. Cells transfected with the NodaMiRNA construct showed dramatically increased levels of apoptosis compared to either control, with dead-to-live ratios rising to almost 4:1. Error bars represent the standard deviation of three separate trials.

FIG. 10: Calibration Curve for Positive-Sense RNA Molecules. 1 g of NodaMiRNA total RNA from an in-vitro transcription reaction was reverse transcribed using NEB MMLV reverse transcriptase, using a reverse primer that targeted the T2A region of the RNA molecule. Varying dilutions of the in vitro transcription reactions were used to generate a calibration plot for the detection of NodaMiRNA RNA in vivo. Ct values were determined by qPCR. Three replicates of each dilution were used to generate the plot, which ranges over 5 orders of magnitude in terms of RNA copy number. This plot was used to calculate the number of NodaMiRNA molecules present in transfected cells.

FIG. 11: MicroRNA Quantification Calibration Curve. 500 ng of mature miRNA, purchased from Integrated DNA Technologies, was initially polyadenylated using NEB poly-a polymerase. The polyadenylated microRNAs were then reversed transcribed using an adapter molecule that hybridized to the 3′ ends of the microRNA, as well as the poly-A tail. The reverse transcription reaction was performed per manufacturer's instructions using NEB MmuLV reverse transcriptase. Dilutions corresponding to 1 ng, 100 pg 10 pg, 1 pg, 100 fg and 10 fg/L were using to create the calibration curve shown above. One μL of each of the dilutions were assayed in triplicate.

### DETAILED DESCRIPTION OF THE INVENTION

In the description of embodiments, reference may be made to the accompanying figures which form a part hereof, and in which is shown by way of illustration a specific embodiment in which the invention may be practiced. It is to be understood that other embodiments may be utilized, and structural changes may be made without departing from the scope of the present invention. Unless otherwise defined, all terms of art, notations and other scientific terms or terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a substantial difference over what is generally understood in the art. Many of the aspects of the techniques and procedures described or referenced herein are well understood and commonly employed by those skilled in the art. The following text discusses various embodiments of the invention.

Viruses whose genomes are positive-sense single-stranded RNA (often referred to as +ssRNA viruses) make up a large portion of mammalian viruses, and count among their ranks pathogenic viruses such as Yellow Fever, SARS, and Dengue. The genomes of such viruses serve directly as messenger RNAs (mRNAs) for the translation of their gene products. Their associated lifecycles begin upon entry into the cytoplasm of the host cell, where the encapsidated mRNA genome is made available to ribosomes. The first gene products translated are the non-structural proteins involved in the replication of the viral genome; they include RNA-dependent RNA polymerases (RdRp) and helicases, which are known collectively as the replicase proteins and serve as primary organizers for replication and later stages of the viral lifecycle.

As disclosed herein, we have discovered that we can exploit the replication strategy of a particular +ssRNA virus, Nodamura Virus (NoV). With its brother species Flock House Virus (FHV), NoV is a member of the two-molecule-genome alphanodavirus genus (Nodaviridae family) of insect viruses whose life cycles have been extensively studied in a broad range of host cells-(Ball 1995)(Miller and Ahlquist 2002)·(Venter and Schneemann 2008). Their replication involves synthesis by the viral replicase proteins of the reverse complement [minus or (−)-sense RNA] of the genomic RNA [positive or (+)-sense RNA], from which new viral genomes are subsequently transcribed. This process is depicted in FIG. 1 for the case of a (+)-sense RNA derived from molecule 1 (RNA1) of NoV in which a gene of interest (GOI) has been inserted at the end of the open reading frame (ORF) of the replicase gene coding for protein A, the NoV RdRp, separated by a T2A ribosome-shifting sequence. NoV molecule 2 (RNA2) codes for the capsid protein of the virus and is also replicated by the replicase proteins. RNA1 is referred to as a replicon because of its ability to be translated directly to give a gene product that binds and replicates it. In their simplest form RNA replicon molecules contain a gene coding for replicase proteins and the 5′ and 3′ untranslated regions (UTRs) that recruit the replicase.

The invention disclosed herein has a number of embodiments. Embodiments of the invention include compositions of matter comprising a self-replicating polyribonucleotide as disclosed herein. Typically in such compositions, the polyribonucleotide comprises a constellation of elements including a polynucleotide sequence encoding a RNA dependent RNA polymerase such as RNA1, which encodes the viral RNA-dependent RNA polymerase of Nodamura virus; a T2A ribosome-shifting polynucleotide sequence; a polynucleotide sequence comprising a first ribozyme; a polynucleotide sequence comprising a cargo polynucleotide; and a polynucleotide sequence comprising a second ribozyme. In such compositions, these elements are disposed in the polynucleotide such that when the polynucleotide is transduced into a mammalian cell the RNA dependent RNA polymerase encoded by the polynucleotide is made, the polynucleotide is replicated, and the first and second ribozymes cleave the polynucleotide such that a polynucleotide fragment consisting of the cargo polynucleotide is generated.

The polynucleotides of the invention can be disposed in different vectors depending upon the context in which they are used. In certain embodiments of the invention, a polynucleotide of the invention is disposed within a NodaT2A vector (see. e.g. Gitlin et al., (2014) Rapid Evolution of Virus Sequences in Intrinsically Disordered Protein Regions. PLoS Pathog 10(12): e1004529. doi:10.1371/journal.ppat.1004529 and Biddlecome et al. (2019) Delivery of self-amplifying RNA vaccines in in vitro reconstituted virus-like particles. PLoS ONE 14(6): e0215031).

In certain embodiments of the invention, the cargo polynucleotide comprises a microRNA. MicroRNAs (abbreviated miRNA) are small non-coding RNA molecule (containing about 22 nucleotides) found in plants, animals and some viruses, that functions in RNA silencing and post-transcriptional regulation of gene expression. The “miRBase database” provides a searchable database of published miRNA sequences that can be used in embodiments of the invention and annotation. Each entry in the miRBase Sequence database represents a predicted hairpin portion of a miRNA transcript (termed “mir” in the database), with information on the location and sequence of the mature miRNA sequence (termed miR). Both hairpin and mature sequences are available for searching and browsing, and entries can also be retrieved by name, keyword, references and annotation. In other embodiments of the invention, the cargo polynucleotide encodes a polypeptide such as a therapeutic protein.

Embodiments of the invention include methods of making the compositions disclosed herein. Typically, such methods comprise forming a polynucleotide, wherein the polynucleotide is formed to include a constellation of elements such as a polynucleotide sequence encoding a RNA dependent RNA polymerase, a T2A ribosome-shifting polynucleotide sequence, a polynucleotide sequence comprising a first ribozyme, a polynucleotide sequence comprising a cargo polynucleotide, and a polynucleotide sequence comprising a second ribozyme. These methods include organizing the constellation of elements in the polynucleotide such that when the polynucleotide is transduced into a mammalian cell the RNA dependent RNA polymerase encoded by the polynucleotide is made, the polynucleotide is replicated, and the first and second ribozymes cleave the polynucleotide such that a polynucleotide fragment consisting of the cargo polynucleotide is generated. A variety of different elements can be used to make such polynucleotides both in vitro and in vivo. For example, in certain embodiments of the invention, the polynucleotide sequence encoding the RNA dependent RNA polymerase encodes a Nodaviridae RNA dependent RNA polymerase such as Nodaviridae RNA1 (see, e.g. Kim et al., Microbiol Biotechnol. 2019 Nov. 28:29(11):1790-179). In illustrative embodiments of the invention, the polynucleotide sequence comprising the first ribozyme or the polynucleotide sequence comprising the second ribozyme comprise a Hammerhead ribozyme and a Tetrahymena ribozyme. Ribozymes useful in embodiments of the invention are described, for example, in Ribozymes and siRNA protocols (Methods in Molecular Biology) 2nd Edition by Mouldy Sioud (Editor) and Ribozymes: Methods and Protocols (Methods in Molecular Biology) 2012th Edition by Jörg S. Hartig (Editor) and United States Patent Application 20030096399. In some embodiments of the invention, the polynucleotide sequence comprising a cargo polynucleotide consists of a miRNA. In other embodiments of the invention, the polynucleotide sequence comprising a cargo polynucleotide comprises an open reading frame of a polypeptide of interest that is expressed in transduced cells. In typical embodiments, the elements in the polynucleotide are selected and organized such that when the polynucleotide is transduced into a mammalian BHK-21 cell, at least 5,000, 10,000, 50,000 or 500,000 polynucleotide fragments consisting of the cargo polynucleotide are generated.

Embodiments of the invention also include methods of delivering a cargo polynucleotide into a mammalian cell either in vitro or in vivo, the method comprising combining the mammalian cell (e.g. a cancer cell) with a self-replicating polynucleotide composition disclosed herein under conditions selected so that the cargo polynucleotide is transduced into the mammalian cell. A variety of conventional methodologies can be used to transduce the mammalian cell. Optionally for example, in in vitro work with cell culture, the cargo polynucleotide is transduced into the mammalian cell via a lipofection method. Typically in these methods, when the cargo polynucleotide is transduced into the mammalian cell, at least 2,000 polynucleotide fragments consisting of the cargo polynucleotide are generated.

Compositions of the invention can be formulated as pharmaceutical compositions in a variety of forms adapted to the chosen route of administration. The compounds of the invention are typically administered in combination with a pharmaceutically acceptable vehicle such as an inert diluent. For compositions suitable for administration to humans, the term “excipient” is meant to include, but is not limited to, those ingredients described in Remington: The Science and Practice of Pharmacy, Lippincott Williams & Wilkins, 21st ed. (2006) the contents of which are incorporated by reference herein.

The compounds may also be administered in a variety of ways, for example intravenously. Solutions of the compounds can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations can contain a preservative to prevent the growth of microorganisms.

The pharmaceutical dosage forms suitable for injection or infusion can include sterile aqueous solutions or dispersions or sterile powders comprising the compounds which are adapted for the extemporaneous preparation of sterile injectable or infusible solutions or dispersions. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof.

Useful liquid carriers include water, alcohols or glycols or water/alcohol/glycol blends, in which the compounds can be dissolved or dispersed at effective levels, optionally with the aid of non-toxic surfactants. Adjuvants such as additional antimicrobial agents can be added to optimize the properties for a given use.

Effective dosages and routes of administration of agents of the invention are conventional. The exact amount (effective dose) of the agent will vary from subject to subject, depending on, for example, the species, age, weight and general or clinical condition of the subject, the severity or mechanism of any disorder being treated, the particular agent or vehicle used, the method and scheduling of administration, and the like. A therapeutically effective dose can be determined empirically using the disclosure presented herein, by conventional procedures known to those of skill in the art. See e.g., The Pharmacological Basis of Therapeutics. Goodman and Gilman, eds., Macmillan Publishing Co., New York, 13^{th} Edition. For example, an effective dose can be estimated initially either in cell culture assays or in suitable animal models. The animal model may also be used to determine the appropriate concentration ranges and routes of administration. Such information can then be used to determine useful doses and routes for administration in humans. A therapeutic dose can also be selected by analogy to dosages for comparable therapeutic agents.

In certain embodiments of the invention, constructs disclosed herein may be used for the preparation of a pharmaceutical composition for the treatment of disease. Such disease may comprise a disease treatable by gene therapy, including cancer. The term “pharmaceutical composition”, as used herein, refers to a composition comprising a therapeutically effective amount of active agents of the present invention and at least one non-naturally occurring pharmaceutically acceptable excipient. Embodiments of the invention relate to pharmaceutical compositions comprising one or more constructs disclosed herein in combination with a pharmaceutically acceptable excipient. The particular mode of administration and the dosage regimen will be selected by the attending clinician, taking into account the particulars of the case (e.g., the subject, the disease, the disease state involved, and whether the treatment is prophylactic). Treatment may involve daily or multi-daily doses of compound(s) over a period of a few days to months.

The terms “pharmaceutically acceptable excipient”, or “pharmaceutically acceptable carrier,” “pharmaceutically acceptable diluent,”, or “pharmaceutically acceptable vehicle,” used interchangeably herein, refer to a non-toxic solid, semisolid or liquid filler, diluent, encapsulating material or formulation auxiliary of any conventional type. A pharmaceutically acceptable carrier is essentially non-toxic to recipients at the dosages and concentrations employed and is compatible with other ingredients of the formulation. Suitable carriers include, but are not limited to water, dextrose, glycerol, saline, ethanol, and combinations thereof. The carrier can contain additional agents such as wetting or emulsifying agents, pH buffering agents, or adjuvants which enhance the effectiveness of the formulation.

The person skilled in the art will appreciate that the nature of the excipient in the pharmaceutical composition of the invention will depend to a great extent on the administration route. In the case of the pharmaceutical compositions formulated for use in gene therapy regimens, a pharmaceutical composition according to the invention normally contains the pharmaceutical composition of the invention mixed with one or more pharmaceutically acceptable excipients. These excipients can be, for example, inert fillers or diluents, such as sucrose, sorbitol, sugar, mannitol, microcrystalline cellulose, starches, including potato starch, calcium carbonate, sodium chloride, lactose, calcium phosphate, calcium sulfate or sodium phosphate; crumbling agents and disintegrants, for example cellulose derivatives, including microcrystalline cellulose, starches, including potato starch, sodium croscarmellose, alginates or alginic acid and chitosans; binding agents, for example sucrose, glucose, sorbitol, acacia, alginic acid, sodium alginate, gelatin, starch, pregelatinized starch, microcrystalline cellulose, aluminum magnesium silicate, sodium carboxymethylcellulose, methylcellulose, hydroxypropyl methylcellulose, ethylcellulose, polyvinylpyrrolidone, polyvinyl acetate or polyethylene glycol, and chitosans: lubricating agents, including glidants and antiadhesive agents, for example magnesium stearate, zinc stearate, stearic acid, silicas, hydrogenated vegetable oils or talc.

Embodiments of the invention include using an RNA replicon with a subgenomic promoter controlling the amplified synthesis of short noncoding RNA that is active in gene regulation via the DICER/RISC pathway. The structure of the Sindbis virus genome is typical of many plus-sense [positive-strand] RNA viruses. In the Sindbis virus genome, the first viral open reading frame (ORF1) is directly translated by ribosomes to produce a polyprotein which, upon self-cleavage and association with host cells factors, serves as an RNA-dependent-RNA-polymerase (RdRp) acting on the original positive-strand RNA molecule to make full-length complementary (minus) strands. Note that elements such as a stop codon just after ORF1 can be used to ensure that only the replicase proteins are synthesized in the primary translation of the viral genome. After buildup of thousands of copies of full-length minus strands, the complex of RdRp proteins undergoes a reorganization and begins to bind to the minus strands instead of the plus strands, making up to hundreds of thousands of copies of both the full-length plus strands and plus-strand copies of the original ORF2. The latter are messenger RNA (mRNA) molecules for the proteins encoded by ORF2. More explicitly, the reorganized RdRp complex binds not only to the minus-strand complement of the 5′ end of the genome, but also to the complement of the subgenomic promoter, thereby making the ORF2 mRNAs. Thus, “transcription” generating this mRNA necessarily depends on—and hence enjoys the powerful amplification benefits of—high-level RNA replication.

Embodiments of the invention can replace a viral ORF2 with an RNA sequence that corresponds to a short noncoding RNA known to knock down gene expression via the dicer/RISC RNA interference pathway. Normally this interfering RNA is synthesized by a much more complicated and less efficient process, namely by transcription of DNA genes in the nucleus, followed by processing and export of the resulting (“primary” micro [pri-micro]) RNA to the cytoplasm, where it is further processed by DICER into “pre”- and “mature”-micro-RNA ready to be picked up by the RISC RNA interference machinery. These latter RNAs are identical to the short-noncoding RNAs that our invention produces directly in the cytoplasm by means of the replication of our RNA replicon, following its translation as described above.

When the requisite genes for RNA replication and the genes coding for target proteins—genes of interest (GOI)—are included in a single RNA molecule, they establish sufficient conditions for replication of the initial RNA molecule within a suitable host cell. Because these molecules do not code for the structural proteins that compose a capsid, they do not represent a true virus or even a viral genome. Instead, they are “just” self-amplifying RNA (saRNA) molecules—replicons. Accordingly, because the replication mechanisms of a replicon are different from those in an active viral infection, we call the former state a “pseudoinfection” insofar as it exhibits some but not all of the traits associated with true viral infection (Lindenbach et al. 2002). Given their ability to replicate to high copy number, RNA replicon systems are exciting candidates for the delivery of mRNAs to mammalian cells: insertion of a gene of interest into the replicon allows for its over-expression concomitant with replication and translation of the replicon (Lundstrom 2016)·(Brito et al. 2015)·(Khromykh 2000). NoV, which naturally infects insects, and its engineered replicons have been shown previously to exhibit robust replication across many cell types and to be attenuated in activity at 37° (Maharaj et al. 2014)·(Johnson and Ball 1999)·(Biddlecome et al. 2019), limiting their cytotoxicity and allowing for targeting tissue-specific tumors that replicate in cooler environments, such as testicular cancers.

The genomes of (+) ssRNA viruses can also be used to deliver functional, noncoding RNAs, such as microRNAs, to the cytoplasm of cells. MiRNAs are short (16-27 nucleotides) single-stranded RNA molecules that are one of the keystone regulatory molecules in the eukaryotic gene regulation network—there are over 2,600 miRNAs in humans (Lim et al. 2003)·(Griffiths-Jones 2010). They have a long half-life and are biologically effective even at low concentrations, which make them ideal candidates for delivery in an amplifying RNA construct. While miRNAs were originally thought to be involved only in sequence-specific, post-transcriptional, gene regulation it is now known that they also play a role in transcriptional silencing and that they interact strongly with a variety of proteins, including tumor suppressor protein p53 (Navarro and Lieberman 2015). These non-canonical interactions enhance the capacity for each miRNA to regulate a myriad of protein targets, increasing their versatility as regulatory molecules.

Particular miRNA species are known to either suppress or induce tumor growth, and altering the number of these regulatory molecules changes the progression and morphology of various cancers (Liu et al. 2011). Downregulation of miR-34a, which belongs to the miR-34 class of miRNAs, is well known to be a major factor in accelerated cancer progression: miR-34a represses the protein SIRT1, which is a key part of the p53 regulatory network (Yamakuchi et al. 2008). miR-34a also reduces the spread of prostate cancer cells by inhibiting the cell-surface adhesion receptor CD44 and lowers the probability of metastasis by inhibiting C-Met translation in carcinoma cells (Li et al. 2009). In addition, beyond decreasing protein synthesis, miR-34a interacts directly with p53 to regulate many different pathways (Chang et al. 2007).

Restoration of homeostatic levels of these miRNAs provides a compelling treatment option for cancer. Unlike the delivery of anti-cancer transgenes, off-target delivery of miRNAs is less deleterious, as the non-cancerous cells already have high levels of miRNA present and would be less perturbed by an additional change in levels. In previous studies pre-miRNA/miRNA molecules have been delivered to cells using a variety of methods (Mansoori et al. 2014). However, these molecules are non-self-replicating, and accordingly their effective delivery must rely on a large initial load of miRNAs to ensure a long duration of the effect. Delivery of self-amplifying miRNAs is therefore an extremely attractive alternative because it removes the requirement of a high input concentration and naturally increases the duration of effect.

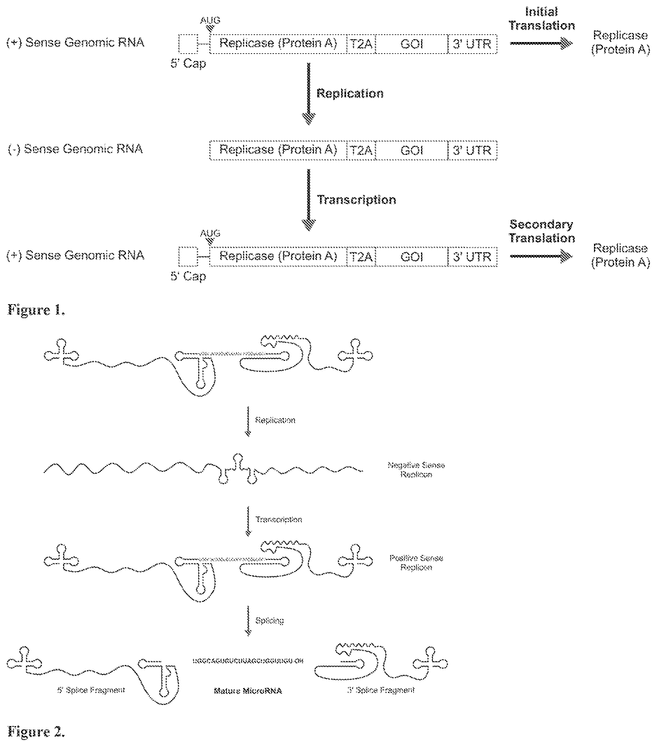

We describe here the synthesis, biochemical characterization, and biological activity of an RNA cassette that contains a coupled system of ribozymes that cleave themselves out of the RNA transcript, resulting in the 22-nt mature miR-34a. In particular, the mature miR-34a sequence is flanked 5′ by a modified hammerhead ribozyme, which cleaves directly upstream of the first nucleotide of miR-34a, and 3′ by a modified Tetrahymena group I intron, which ensures the miRNA 3′-end is hydroxylated (FIG. 2).

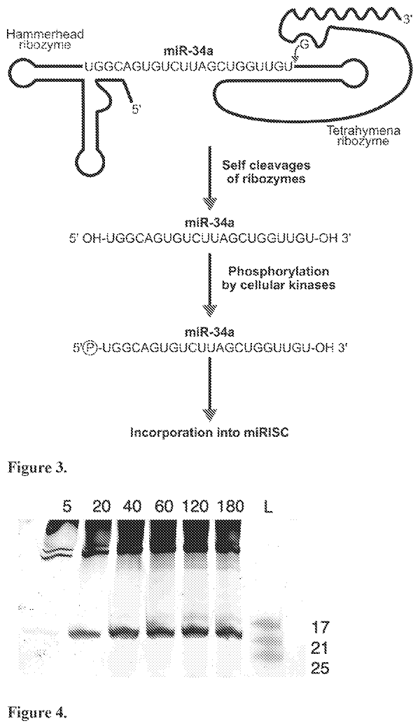

When incorporated into the NodaT2A replicon (replacing the GOI in FIG. 1, shown in FIG. 3), this cassette is strongly replicated and actively cleaved in BHK-21 cells, yielding on the order of one million miR-34a molecules per cell. These miRNAs are shown to be biologically active, inducing essentially complete apoptosis in cultured prostate cancer (PC3) cells.

### The Core Catalytic Sequence—Ribozyme-Flanked MiRNA—Cleaves Co-Transcriptionally

To assess the ability of the designed construct to cleave appropriately, a co-transcriptional time course was performed to measure the accumulation and cleavage of the RNA molecules. FIG. 4 shows the gradual accumulation of 22-nt RNA during the in vitro transcription of the 470-nt-long Core Catalytic Sequence (CCS) template. At the specified times, aliquots from the in vitro transcription reaction were denatured and loaded onto a 20% acrylamide, 8M urea gel. A strong band at 22-nt appears faintly at 5 minutes and gradually accumulates over the course of the transcription reaction. After about 1 hour the band's intensity appears to level off, indicating that either the in vitro transcription reaction has stopped, or all of the RNA has cleaved. When the reaction is run on a 12% Urea PAGE gel, both full-length precursor molecules as well as the 5′ and 3′ cleavage products are visible and run at the appropriate positions (not shown). The presence of both these bands, increasing in intensity with time, coupled with the increasing intensity of the band running at 22 nt, provides strong evidence that both ribozymes are cleaving effectively and that they are doing so in a co-transcriptional manner. It appears that the complicated secondary/tertiary structures present in both ribozymes do not affect each other, even though they are close in the primary sequence. The reaction conditions of in vitro transcription are sufficient for cleaving of both the ribozymes; the mix contains high levels of magnesium ions, as well as the guanosine cofactor needed for cleavage of the group-I intron. The large majority of the RNA cleaves quickly upon transcription, with little to no full-length RNA present in the mix after 2 hours. The intense bands present at the top of the gel are the 5′ cleavage products, as well as full-length molecules, which cannot be resolved on the gel.

Cleavage Occurs In Vitro when CCS is Incorporated into NodT2A

After verifying that the CCS was capable of efficient cleavage, it was cloned into the NodaT2A vector and we determined the ability of this new molecule to cleave during transcription. The resulting plasmid was linearized, and an in vitro transcription reaction was performed. Aliquots were taken at six time points and were immediately denatured before loading onto a 1.2% denaturing formaldehyde agarose gel: see FIG. 5, with lanes 1-6 corresponding to the aliquots collected from the transcription mix at indicated times.

FIG. 5 shows two strong bands, one running at approximately 3.5 knt (*, corresponding to the full-length Nodamura replicon RNA) and one at 3 knt (**, corresponding to the 5′ cleavage product of the replicon). There is also a faint band at approximately 600 nt (***), which corresponds to the 3′ cleaved fragment. The intensity of the band associated with the full-length molecule is always markedly less than that of the cleaved bands. While the intensity of the cleaved band increases steadily, the amount of the full-length molecule remains almost unchanged, indicating either a subpopulation of RNA too misfolded to be catalytically active or that competing transcriptional and cleavage reactions produce a steady state for the numbers of full-length NodaMiRNA.

### NodaMiRNA RNA is Capable of Establishing Pseudoinfection and Cleaves in Cultured Cells

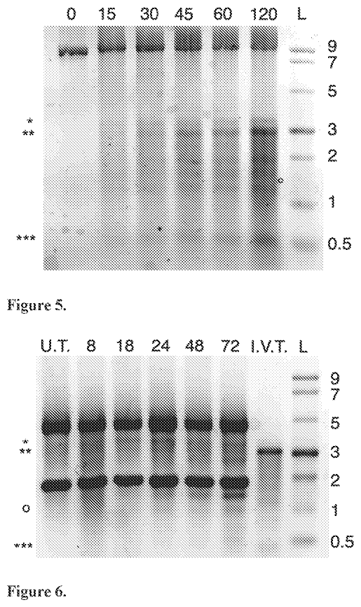

We subsequently tested the replication and self-cleavage of NodaMiRNA-CCS in cells. The NodaMiRNA replicon RNA accumulates in transfected BHK-21 cells to levels far surpassing all other cellular mRNAs (FIG. 6). The full-length (un-cleaved) replicon band, which runs just a bit faster than the larger (28S) rRNA subunit, is clearly visible at 18 hours, and increases in intensity, peaking at around 24 hours. The smear seen around 500-1500 nt represents all other mRNA present in the cell; as the replicon band is more intense than this band, it is clear that replicon RNA accumulates to levels higher than even abundant mRNAs (such as actin or GADPH.) It should also be noted that any minus-strand RNA that accumulates during this time would also be 3.5 knt in length and is therefore indistinguishable from full-length NodaMiRNA RNA when run on a gel. This is because replication proceeds first via the synthesis of the reverse complement of the replicon molecule, which by definition is the same length as the plus-strand genome. However, in NodaMiRNA this minus-strand RNA does not contain active ribozymes and thus does not cleave. Both the 5′ and 3′ cleavage products are clearly visible in the gel the 5′ (**) around 3 knt and the 3′ (***) around 600 nt. In addition to these bands, a band at 1.1 knt (o), which represents the replication of the subgenomic RNA that occurs during the replication cycle, is present from the 18 up to the 72-hour time points.

### NodaMiRNA Replicon RNA Replicates to High Copy Number in Cells

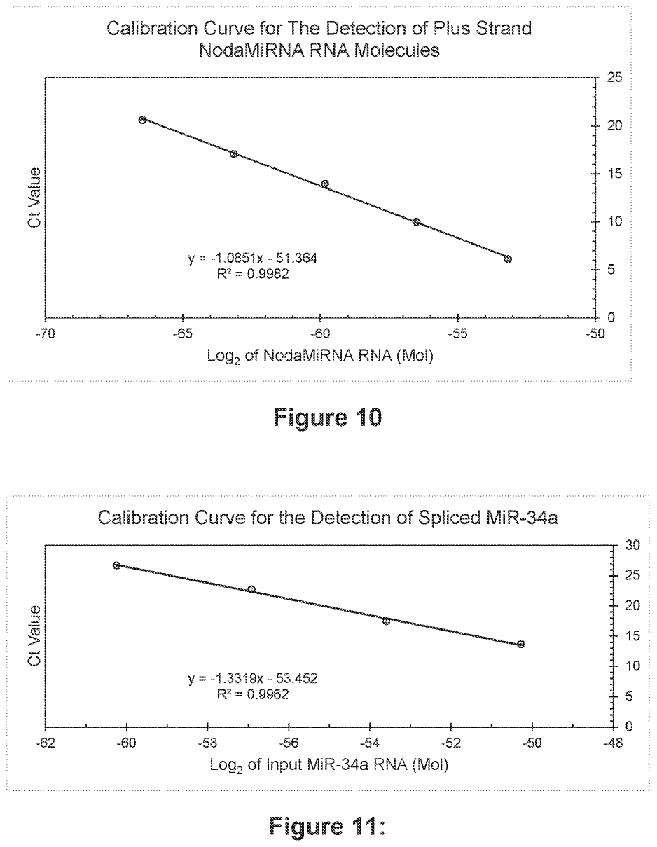

In order to understand the extent of cleavage in cultured cells, it was important to be able to quantify the total number of replicon RNA molecules produced during a pseudoinfection. To do this, a calibration plot was generated using in vitro transcribed NodaMiRNA RNA. This plot, which is shown in FIG. 10, was used to determine the absolute number of replicon molecules present in transfected cells. Primers were designed to anneal to the T2A region of the NodaT2A replicon RNA, which has limited secondary structure and serves as an ideal amplicon region.

RT-qPCR analysis of total extracted RNA from cells transfected with NodaMiRNA replicon RNA shows high levels of RNA accumulation as can be seen in FIG. 7. By 18 hours post-transfection, the replicon has generated as many as 20,000 RNA molecules per cell. This number vastly outnumbers even the most plentiful natural mRNA within the cell, such as actin with about 1,500 copies per cell (Guo et al. 2015). The number of replicons per cell gradually declines, but at 72 hours post-transfection still remains above 5,000.

miR-34a is Produced by NodaMiRNA in Mammalian Cells

We next measured the abundance of miR-34a using quantitative RT-PCR. We first produced a calibration curve using synthetic miR-34a (FIG. 11). The curve is linear and has very good regression statistics, although the slope is a bit high, implying that the mature microRNA detection threshold for this curve is less than that for the full-length replicons. The assay specificity was measured via melt-curve analysis, which resulted in a single peak.

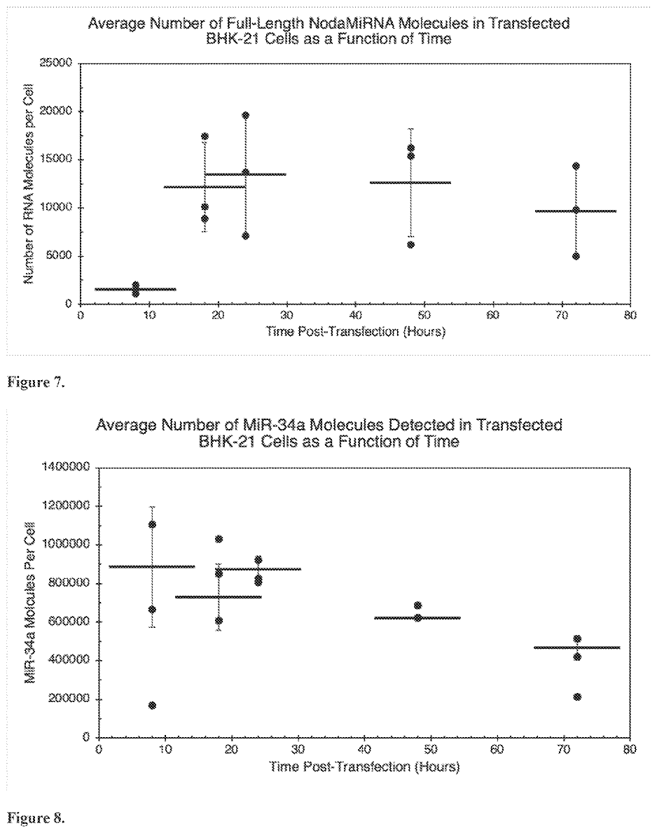

FIG. 8 shows the absolute number of microRNAs detected using RT-qPCR from cells transfected with NodaMiRNA replicons. The expression profile of miRNAs produced by NodaMiRNA has the same general shape as the expression profile for NodaMiRNA itself. Initial miR-34a expression is relatively high, likely due to early cleavage events of the delivered full-length NodaMiRNA and its replication products, with time points 18 hours post-transfection and later mirroring the trend seen in the full-length expression time course: MiR-34a expression again peaks at 24 hours post-transfection and decays slowly thereafter. It should be noted that the miRNA levels greatly exceed the replicon RNA levels at later time points, indicating that the replicon is cleaving efficiently in cells. This is because the miRNAs have very long half-lives in cells (>48 hours) compared to the replicon RNA, which is viral in composition and actively destroyed by the cell.

Replicon-Produced miR-34a is Biologically Active in PC3 Cells

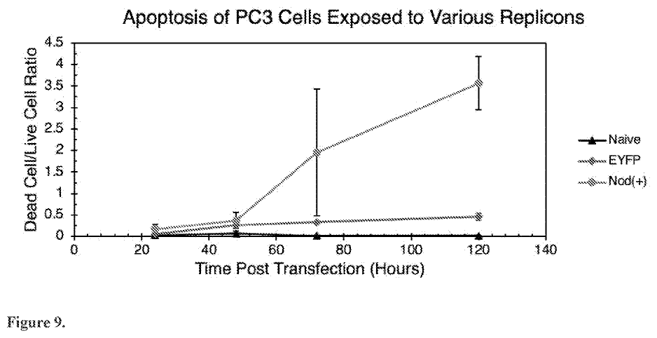

By calculating the ratio of dead to live cells transfected with NodaMiRNA or a control EYFP replicon (with EYFP in place of the CCS in the Nodamura replicon) versus naïve (untransfected) cells, a useful metric is generated for determining the effect of the replicon on the cell viability (FIG. 9). Use of Trypan Blue allows for the detection of dead cells in culture, and for direct probing of the replicon's cytotoxic effects. Dying cells are stained blue by the dye and are easily counted during the measurement of viable cells. Untransfected cells demonstrated an almost constant dead/live ratio of 0.09. Cells transfected by a control EYFP replicon showed a steadily increasing dead/live ratio, starting at 0.04 at 24 hours, and ending with a value of 0.5 at 120 hours. The curve generated by NodaMiRNA is functionally 15 different from those of either the mock-transfected cells or the EYFP-transfected cells. The cells transfected by NodaMiRNA show ever-increasing levels of dead cells, with a final dead/live ratio of about 3.5, approximately 7 times greater than the EYFP control, and 40 times more than mock-transfected PC3 cells. Previous assays with BHK-21 cells indicated that the replicon was not cytotoxic, so any increased cytotoxicity must be tied to either the cell type transfected or the biological activity of the cleaved microRNA.

The gel depicting the in vitro cleavage of the CCS (FIG. 4) demonstrates the high efficiency of the ribozymes outside of the cellular environment. When subsequently incorporated into the NodaMiRNA replicon system, the co-transcriptional cleavage of the ribozymes is somewhat attenuated (FIG. 5). The reaction conditions for the initial in vitro transcription reactions of the CCS alone and for the subsequent Message transcriptions are similar in magnesium and guanosine concentrations, so it is unlikely that the differences in cleavage efficiency can be attributed to differences in buffer conditions. However, both the hammerhead and group-1 intronic ribozymes require their RNA sequences to adopt a specific secondary/tertiary structure in order to function. The insertion of the CCS into the larger replicon structure undoubtedly allows for the formation of non-catalytic secondary structures between nucleotides present in either ribozyme and in sequences directly adjacent. Some of these alternatively-folded RNAs were stable enough to initiate pseudoinfections, as seen in FIG. 6.

A striking aspect of FIG. 6 is the level of RNA accumulation in transfected cells. Replicon RNA accumulates to levels higher than typical mRNAs, which indicates that the small number of full-length replicon RNAs delivered to cells is sufficient to produce a robust pseudoinfection. Ribozymes are usually more catalytically active in vivo than in vitro, owing partially to the crowded nature of the cytoplasm that helps them fold into their active configurations (Paudel and Rueda 2014). FIG. 6 confirms this, as the intensity of the cleaved RNA band, which runs at 3 knt, is plainly visible at 18 hours, and reaches levels similar to that of the full-length genomic RNA (3.5 knt). The nature of viral replication aids in the cleaving of the ribozymes: replication of viral RNA requires conditions similar to those of in vitro transcription. The high levels of magnesium and guanosine needed in the synthesis of genomic RNA aid in the cleavage of the RNA and the production of active miR-34a. It should be noted that the band at 3.5 knt represents not just the full-length NodaMiRNA RNA, but also the negative strand RNA that is generated during an active infection, and thus this band is expected to always be larger than the band that represents the cleaved replicon. Quantitation of minus-strand RNA via qPCR indicates that it accumulates at a level similar to that of positive strand (not shown).

The RNA levels determined using qPCR match the general trend seen in FIG. 6, as well as in other reports (Ball et al. 1992). The number of RNA molecules, shown in FIG. 7, peaks around 24 hours, and decays moderately. Wild-type Nodamura infections are much more persistent than infection by the NodaMiRNA replicon, likely because the replicon does not produce the B2 protein that is a known inhibitor of RNAi and that helps the cell mediate the levels of viral RNA during infection (Johnson et al. 2004). Without active B2 expression, the cell is more capable of eliminating the exogenous RNA, attenuating the accumulation of replicon RNA and shortening the time course of the pseudoinfection.

The accumulation of miRNAs derived from the cleavage of the CCS follows a trend similar to that of the full-length NodaMiRNA genome after an initial 8-hour peak likely due to early cleavage of delivered NodaMiRNA. It should be noted that even at 72 hours there are still >200,000 miRNAs per cell (assuming 1.2×10⁶ cells), which is far above the concentration regime of even high-copy native miRNAs in tissues. The nature of the assay removes any dead, non-attached, cells; the values presented here represent the RNA levels in healthy cells. At the height of the infection, the number of miRNAs is of order a million in each cell: again, surpassing the maximum of miRNAs found in most human tissues. MiR-34a is known to have a half-life >48 hours, which given the closely-spaced time points also explains the large amount of RNA that accumulates during the course of the pseudoinfection, as well as the high copy number at later time points. Even if the full-length NodaMiRNA RNA is degraded by cellular ribonucleases, and as such not detectable by qPCR, the miRNA produced by the cleavage persists in the cell.

While the NodaMiRNA construct indeed replicated readily within cells, and cleaved efficiently, it was uncertain if the nascent miRNA would be biologically active. The 5′-end of the small RNA produced by ribozyme self-cleavage contains a 5′-OH (due to the action of the hammerhead ribozyme) and must be phosphorylated before it becomes fully mature. It was unknown if cytoplasmic kinases would phosphorylate the RNA after cleavage; however, work by Martinez, et al. (Martinez et al. 2002) indicates that exogenous ssRNAs are phosphorylated in cells, and as such we expected similar results. Moreover, there is a direct interaction between the Dicer and the RISC complex, and it was not known if these ssRNA molecules would be subsequently active in regulating target genes. However, because miR-34a is involved not just in the RISC pathway, but in the p53 regulatory network as well as the SNAIL pathway (Hayes et al. 2014), there are alternative avenues for regulation of the cancerous state outside of canonical post-translational silencing. Transfection of PC3 cells with NodaMiRNA RNA demonstrates the biological activity of the miRNA produced by the cleaving of our construct. FIG. 9 shows the ratio of dead to live cells present as a function of time after transfection with 0.5 μg of NodaMiRNA RNA or NodaEYFP replicon RNA. The untransfected cells remain constant at approximately 95% viable cells. For the cells transfected with NodaEYFP, the percentage of viable PC3 cells dips only to about 75%. While this number is indeed less than that of the naïve cells, it is still much greater than those cells treated with NodaMiRNA RNA, where the viable percentage falls steadily to about 20%.

Small-RNA-based therapies, like aptamers (Gold et al. 2012), have become a major focus in biologics development in recent years due to their specificity in targeting proteins implicated in disease and the broadly applicable nature of their design (Nimjee et al. 2005). However, as with these and all gene therapies, a fundamental issue with replicon-based strategies is that the active component must pass through the cell membrane and enter the cytoplasm to be translated in a cell-specific manner. In addition, these molecules must in some way be protected from the abundant ribonucleases present in vivo. A variety of liposomal- and polymer-based systems have been used to deliver mRNAs to cells, but these complexes are highly polydisperse and difficult to characterize, which does not make them ideal candidates for treatment (Tros de Ilarduya et al. 2010). Moreover, due to the transient nature of mRNAs within cells, many RNA molecules must be delivered to each cell, requiring numerous large complexes to be delivered (Leonhardt et al. 2014). Replicons, however, are unique in that they are self-amplifying: one needs only to deliver (in an ideal situation) one molecule to the cell for it to be effective. This changes the paradigm of targeting to focus on the delivery of a single, monodisperse, particle containing a single replicon molecule. A delivery system developed in our lab involves the in vitro reconstitution of spherical virus-like particles composed of bromovirus capsid protein protecting the modified genome of Nodamura (Biddlecome et al. 2019): these chimeras are active in mammalian cells and have their RNA replicated, and are also effective in eliciting an immune response in mice toward their gene-antigen of interest, indicating that they are taken up by antigen-presenting cells and that their RNA is replicated sufficiently to induce T-cell activation (Biddlecome et al. 2019). Similar virus-like-particles can be used as a platform for in vivo delivery of self-replicating miRNAs of the kind presented in this work.

Quite generally, positive-sense RNA viruses, and the replicons derived from them, represent potent and novel vectors for the delivery of functional nucleic acid to cancerous cells. Replicons in particular are ideal as they are non-infectious and lack many of the immunogenic proteins associated with the full-length virus. The nature of their genomes makes them perfect for the delivery of functional RNA and it is a testament to the robustness of the Nodamura system that one can incorporate highly-structured, catalytically-active ribozymes into the replicon molecule without altering the nature of the replication cycle. These insect replicons may represent an excellent compromise between versatility and specificity, owing to their tolerance to large changes in their genomic structure (allowing for incorporation of a diverse set of trans-effectors) and their ability to replicate in a variety of host cells.

### Illustrative Materials and Methods

### Synthesis and Analysis of the Core Catalytic Sequence

The wild-type sequences for both the group-1 intron Tetrahymena, with a tetraloop in place of the wild-type P10 helix (Price et al. 1995), and the hammerhead ribozyme, omitting the 5′ pentaloop (Guo and Cech 2002), were obtained and engineered to auto-catalytically cleave themselves out of a transcript, yielding a 22-nt, mature, miRNA (miR-34a). The modified sequence was purchased from GenScript with appropriate restriction sites for later cloning. The annotated full-length DNA sequence can be found below (in Table S1 below). The design of this cassette is shown in FIG. 2.

The 470-bp DNA template used for the initial in vitro transcription reactions was generated using PCR to add the full T7 promoter to the 5′ end of the CCS DNA sequences. The resulting 470-nt blunt-end product was purified with a Qiagen Qiaquick column and used for subsequent in vitro transcription reactions.

Initial in vitro transcription reactions were performed using 0.2 μg of purified DNA template in MMessage MMachine kit reaction conditions. During transcription, 2 μL aliquots of the transcription mix were taken at several time points over the course of 3 hours and immediately added to 5 μL 100% formamide and heat denatured at 65° C. for 5 minutes prior to loading onto a 20% acrylamide, 8M urea PAGE gel. The gel was pre-run in 1× TBE at 20W for 45 minutes, then run at 8W for 40 minutes. It was then rinsed with ddH₂O and stained for 10 minutes with SYBR Gold (Thermo Fisher) prior to imaging.

Cloning of the CCS into the NodT2A Vector

The DNA sequence corresponding to the CCS insert was sub-cloned out of its initial PUC18 vector via digestion with AgeI and NdeI (New England BioLabs). This insert was purified and ligated into the NodaT2A replicon (see FIG. 1) and designed by Gitlin et al.⁸, with the CCS taking the place of the GOI. The replication scheme for this molecule is depicted in FIG. 3.

The presence of the target sequence in the Nodamura replicon vector was determined by both restriction digest (using AgeI and NdeI) as well as sequencing. This vector (known as NodaMiRNA) is used to generate the full-length Nodamura replicon, containing the CCS, for in vitro transcription studies and transfection experiments.

### In Vitro Cleavage Assay of NodaMiRNA

The above plasmid was linearized via restriction site BsmBI (New England BioLabs), upstream of the HDV ribozyme normally included in the 3′UTR of the Nodamura replicon, and replicon molecules were generated via in vitro transcription with an Invitrogen MMessage MMachine T7 transcription kit. 6 μL aliquots were taken at six time points (t=0, 15, 30, 45, 60, 120 minutes). The samples were immediately denatured in 6 μL 100% formamide at 65° C. for 5 minutes. Samples were frozen at −80° C. until loading onto a 1.2% denaturing MOPS-formaldehyde agarose gel and run at 80 V for 2 hr. Gels were stained for 10 minutes with SYBR Gold (Thermo Fisher) and imaged using a UV transilluminator.

### Transfection of BHK-21 Cells

BHK-21 cells from ATCC were split 24 hours prior to transfections and grown to 70-90% confluence in 6-well or 24-well plates. Cells were transfected with 2 μg of RNA (6-well) or 0.5 μg of RNA (24-well) per well using Lipofectamine 2000 (Thermo Fisher), according to manufacturer's specification, using 10 μL of Lipofectamine per well (6-well) or 2 μL (24-well). The transfection mixture was overlaid onto the confluent BHK cells, and the cells were incubated for 8 hours at 37° C. in 5% CO₂, unless otherwise indicated. After 8 hours of transfection, the transfecting medium was removed from the cells, the cells were washed with 2 mL warmed PBS, and the cells were subsequently overlaid with DMEM supplemented with 10% Fetal Bovine Serum (FBS). This medium was left on top of the cells for the remainder of the experiment.

### Generation of Positive-Strand Calibration Curves

In order to accurately quantify the total number of positive and negative strands generated during replicon replication, a calibration curve was generated to determine the total number of each RNA from the total purified cellular RNA. 1 μg of in vitro transcribed NodaMiRNA RNA was reverse transcribed using NEB MMULV reverse transcriptase per manufacturer's specification. The RNA region chosen for detection was the 140-nt region spanning from 40 nt upstream of the T2A peptide, through the T2A coding region. This region was chosen because of the relatively small extent of RNA secondary structure, as predicted by Mfold (Zuker 2003). All relevant primers for Nodamura detection are presented in Table S2 below. After reverse transcription the reverse transcriptase was heat-denatured at 65° C. for 20 minutes, and a series of dilutions was generated by serially diluting the reverse transcription mixture, at 50 ng/μL, with ddH₂O, yielding the following concentrations: 1 ng, 100 pg, 10 pg, 1 pg, 100 fg, and 10 fg/μL. 1 μL of each of these dilutions was used in the generation of the calibration plot.

The calibration curve was generated using a Pharos Opticon fluorescent thermocycler. Each calibration point was done in triplicate, and consisted of 1 μL of the relevant dilution, 10 μL of Bio-Rad Ssoadvanced qPCR master mix, 200 nM each forward and reverse primer, and water to 20 μL. These samples were loaded onto a 96-well, optically clear plate, sealed with transparent film and analyzed. Polymerase chain reaction was performed with an extension temperature of 60° C. for 30 seconds, during which time the fluorescence emission of the SYBR green dye was measured. Cycle threshold (Ct) values corresponding to each dilution in the series were measured using the Opticon software and exported for analyses. The Ct values for each of the 6 dilutions were averaged and plotted against the log of the input RNA, yielding a linear calibration curve. This curve was used to quantify the number of Nodamura molecules present in the total cellular RNA samples taken at various time points post-transfection.

### Harvesting and Analysis of Total Cellular RNA

At several time points (8, 18, 24, 48, and 72 hours post-transfection) the total cellular RNA was harvested from transfected cells using Qiagen RNeasy columns, according to manufacturer's specification. RNA was eluted from columns using 35 μL of RNase-free water, and the concentration of RNA was determined by UV absorption using a Nanodrop spectrophotomer. The purified RNAs, containing rRNA, cellular mRNA, and full-length and cleaved Nodamura replicon RNA were used for downstream analysis. Total cellular RNAs were also purified from naïve BHK-21 cells as well, using the techniques described above. RT-qPCR was used to analyze the cellular extracts, with 1 μg from each well reverse-transcribed using the MMULV system as above. 100 μg of the resulting cDNA was added to 10 μL of Bio-Rad Ssoadvanced qPCR master mix, 300 nM each of forward and reverse primer, and water to 20 μL. The resulting Ct values were analyzed using the equation generated from the calibration curve to determine absolute RNA mass generated. All time points were collected in biological triplicate and assay triplicate.

RNA numbers per cell were estimated using the conversion calculator from molbiol.edu to convert RNA mass (acquired from calibration curves) to RNA numbers, dependent on the length of the species in question. The resulting value was then divided by 1.2×10⁶ cells per confluent 6-well plate, as per Thermo-Fisher's “Useful Numbers for Cell Culture” approximation.

### Gel Electrophoresis of Purified Cellular RNAs

1 μg of total cellular RNA from each time point was denatured in an equal volume of formamide. The RNA was denatured at 65° C. for 10 minutes, loaded onto a 1.2% agarose gel, and run at 75 V for 2 hours. The gel was then stained with GelRed nucleic acid stain for 30 minutes, prior to imaging.

### MiRNA Calibration Plot

To generate the miRNA calibration plot, 900 ng of miR-34a was first polyadenylated using 20 units of NEB polyA-polymerase supplemented with 10 mM ATP in MMULV reverse transcriptase buffer (in which polyA-polymerase is fully active) in order to avoid buffer exchange when starting the reverse transcription reaction (Shi and Chiang 2005). This reaction was run at 37° C. for 1 hr. An adapter molecule that anneals to the polyadenylated 3′ end of the microRNA was added to this mixture to a concentration of 50 μM, and the mixture was diluted with water to a final volume of 30 μL. The resulting mixture was heated to 65° C., then cooled on ice for 5 minutes. Additional MMULV buffer (2 μL) and MMULV reverse transcriptase (2 μL) were added to the tube, as well as water, to a final volume of 40 μL. The reaction was run at 42° C. before being diluted to the following concentrations of original RNA input mass: 1 ng, 100 pg, 10 pg, 1 pg, 100 fg, and 10 fg/μL. One μL of each of these dilutions was used in the creation of the calibration plot as above, with primers annealing to sequences within the microRNA.

Purification and Analysis of Cellular microRNAs

The small fraction of RNAs, consisting of RNAs <100 nts, was purified at specific time points from transfected BHK-21 cells using the Qiagen MiRNeasy and MinElute RNA purification systems following manufacturer's specification. This process ensured that only fully cleaved miRNAs were purified from the total cellular milieu. The small RNAs were subjected to Rapid Amplification of cDNA Ends (RACE) analysis by first being polyadenylated using polyA-polymerase supplemented with 1 μL 10 mM ATP for 1 hour at 37° C. The adaptor molecule was then added as in the construction of the calibration plot, and the RNAs were reverse transcribed for 1 hour at 42° C. using MMULV reverse transcriptase. The resulting cDNA was analyzed using RT-qPCR with primers specific to the microRNA in question. A table of the primers used for RT-qPCR can be found in the Supplemental Information (Table S2). The Ct values measured for these samples were then converted to absolute RNA mass using the calibration plot described above, and to absolute RNA numbers per cell using the estimation protocol previously described. These values were collected in assay triplicate and biological triplicate to ensure robust statistics. The qPCR reaction was run on a 1.2% agarose gel in TAE for 1.5 hours prior to being stained with GelRed nucleic acid stain. The reaction ran as a single band at approximately 90 bp, indicating the reaction produced no off-target products.

Cell Proliferation Assay PC3 cells were cultured in F12K medium supplemented with 10% FBS and split into 24-well plates at approximately 10% confluence. These cells were then transfected for 18 hours with 1.5 μg of NodaMiRNA or Nodamura EYFP replicon RNA, using Lipofectamine 2000 following manufacturer's instruction. As a control, cells were mock-transfected using only the transfection reagent with no replicon RNA present. The transfected cells were placed in a 30° C., 5% CO₂ incubator for the initial 36 hours, after which they were transferred to the 37° C. incubator. The cells were moved from the 30° C. incubator to the 37° C. 5% CO₂ incubator to allow for the initial replication of the RNA (which is much stronger in PC3 cells at 30° C. than at 37° C.) without affecting the cells' overall growth profile over the duration of the experiment, because the cells grow much faster at 37° C. than at 30° C. Fluorescence images of transfected PC3 were used to determine when the replication in EYFP-replicon-transfected cells was occurring.

At several times post-transfection, cells were collected and counted using a hemocytometer. The replicon can induce infected cells to become semi-adherent, and as such the cell-collection protocol was modified to ensure collection of all cells. The medium on top of the cells, as well as the PBS used to wash the cells, was collected in addition to the cells that were removed from the plate using trypsin. This is particularly important for PC3 cells, as several of the cells' phenotypes are only weakly adherent, a trait exacerbated by the activity of the replicon. The collected cells were pelleted gently in a centrifuge for 5 minutes at 200×g and resuspended in identical volumes of media supplemented with Trypan Blue stain. 10 μL of this resulting mixture was added to a hemocytometer, and the total number of live and dead cells was measured. The effect of the replicon on the cells was determined two ways: by measuring the proliferation of the transfected cells, and by measuring the number of dead cells with Trypan Blue exclusion.

As noted above, small RNAs have great potential in cancer treatment. Key miRNAs are often mis-regulated and correction of their expression mitigates and in some cases even reverses oncogenesis. siRNAs can routinely be identified to potently and specifically target oncogene expression. While RNA interference has revolutionized our understanding of gene regulation and inspired many new approaches to the treatment of viral disease, cancer, and other genetic disorders, efficient and specific delivery of therapeutic small RNAs has been difficult to achieve. There is a large amount of literature on the molecular/cellular biology of siRNAs and miRNAs for oncogene knockdown, in vitro and in vivo, and on the many different platforms that have been developed for their delivery. Among other methods, delivery vehicles for interfering RNAs have included ones based on their complexation or conjugation to liposomes, biocompatible polymers, peptides, and aptamers. There are also many delivery systems involving retro-virus and DNA-virus vectors, and plasmids, for getting the DNA sequences into target cell nuclei where transcription yields primary miRNAs to be processed and exported to the cytoplasm for pick-up by the RNA-silencing machinery.

Embodiments of the invention are qualitatively different, aiming to deliver interfering RNA in a form that is replicated to a high level, directly in the cytoplasm, before becoming active in translation repression. Specifically, amplification of the miRNA will be facilitated by incorporating it into a virus-derived RNA replicon form, as described below. This approach involves exploiting the unique RNA replication strategy of plus-sense RNA viruses like Sindbis whose genome has a well known structure. Only the first open reading frame (ORF), coding for the RNA-dependent RNA polymerase (RdRp) proteins, is translated from this molecule, because of a stop codon at the 31 end of the ORF. The RdRp proteins, in association with host cell factors, form a replicase complex that makes up to thousands of minus-strand copies of the full genome. The replicase complex then reorganizes and begins to recognize only the minus-strand templates. But it binds now to two sequences, one in the complement of the 5′ untranslated region (UTR) and the other in the complement of the inter-ORF noncoding region. Consequently, hundreds of thousands of copies of each of two plus-strand RNAs are synthesized. One is the full-length viral genome, of which many copies are needed so that a large number of new virus particles can be synthesized by the host cell. The other is a “subgenomic” RNA. Note that because the subgenomic RNA—the messenger RNA for the structural proteins—is transcribed from minus strands, the associated protein synthesis is dependent on RNA replication and enjoys accordingly the great benefit of high-level amplification (3×10⁵-fold in the case of Sindbis). We can simply replace the structural protein ORF by the sequence for a mature miRNA that targets an oncogene of interest. (The mature miRNA contains a seed sequence complementary to the oncogene mRNA, resulting in its degradation and/or translation repression).

Using the disclosure provided herein, artisans can prepare a self-replicating RNA molecule—replicon—designed to ensure the amplification of a mature miRNA for targeting multiple oncogenes. The most direct way to do this is to excise the Sindbis structural-gene ORF and insert in its stead the sequence of a mature miRNA of interest. An illustrative working embodiment is the miR-34 family of miRNAs, including miR-34a, miR-34b and miR-34c, which are master tumor suppressors. miR-34 simultaneously antagonizes many different oncogenic processes by regulating the expression of well-known proto-oncoproteins that are attractive drug targets themselves. For example, miR-34 regulates the cell cycle by targeting cyclins and cyclin-dependent kinases, contributing to WNT signal transduction and metastasis by repressing the expression of WNT, P-catenin, and Notch, etc. The expression of miR-34 is often pronouncedly reduced in a wide range of solid and hematological malignancies, including cancers of the prostate, lung, breast, pancreas, kidney, liver, skin, bladder, colon, brain and the lymphoid system. Aberrant expression of miR-34 is caused by either genetic or epigenetic changes; the gene loci of miR-34a and of the miR-34b-miR-34c polycistron are located at fragile sites that are frequently altered in cancer, and their promoters are often hypermethylated. miR-34a is transcriptionally activated by p53, whose expression and function are reduced in a large number of cancers. Importantly, reintroduction of miR-34 has been extensively demonstrated to inhibit the development of these cancers, inspiring many therapeutic strategies.

Because the subgenomic RNA sequence in Sindbis flanks the ORF sequence with the full 3′ UTR and an extra 18 nucleotides on its 5′ end, we will flank the mature miR-34a sequence with self-cleaving ribozymes. In one design, we choose to use a Hammerhead ribozyme at the 5′ and the Tetrahymena ribozyme at the 3′. The cleavage by the guanine cofactor of the Tetrahymena ribozyme produces a precise 3′ end identical to natural miR-34a (14). The self-cleavage of the Hammerhead generates a miR-34a with 5′-hydroxyl, which is expected to be efficiently phosphorylated by cellular kinases before miR-34a is incorporated into the effector miRNA induced silencing complexes (miRISC).

Construction of the miR-34a replicon can be checked by sequencing. The self-cleaving activity of the ribozymes can be checked by Northern blot analysis of the cleavage products. The initial Sindbis RNA genome can be produced using in vitro transcription. The transcription can be performed under conditions in which the self-cleavages of the ribozymes are not efficient. The T7 RNA polymerase, nucleotides and salts can be removed and the RNA can be transfected to cultured cancer cell lines. The design of our small RNA expression cassette takes advantage of the fact that the cleavages by the ribozymes can be quite efficient but often incomplete in cells. Therefore, a fraction of the initial replicons remains intact and is available to be replicated by the replicase complex following primary translation of the first ORF. But this fraction can be strongly replicated (up to one-thousand-fold), and the resulting full-length minus strands can be stable against ribozymal self-cleavage. Accordingly, as in the natural viral life cycle, large numbers of full-length plus strands and subgenomic plus strands will result. The latter will contain active ribozyme sites flanking the mature miRNA sequence, and hence will yield large numbers of the mature miRNA of interest.

ILLUSTRATIVE ASPECT 1: Artisans can transfect prostate cancer cell lines with the above described replicons to test their efficacy for the in vitro regulation of CD44. A Sindbis replicon-based system can produce miR-34a as designed; and validated if the miRNA is functional in regulating target gene expression. Artisans can initially perform these tests in prostate cancer cell lines because of the prominent role miR-34a plays in their pathogenesis.

It has been demonstrated that miR-34a inhibits cell migration and invasion by down-regulation of CD44 expression in prostate cancer cell lines and prostate cancer stem cells (15). Synthetic miR-34a duplex can be used as a positive control. A replicon containing an unrelated sequence can be used as a negative control. Artisans can extract total RNAs from cells at various time points post transfection. The RNAs can be analyzed using Northem blotting, qRT-PCR and sequencing analyses. 10 Northern blotting using a 32P-labeled oligonucleotide antisense to miR-34a will indicate all forms of the replicon that contain the miR-34a sequence, including the (+) strand replicon RNA, the (+) strand subgenomic RNA, the subgenomic RNA with one or the other ribozyme cleaved off, and the mature miRNA. To separate these RNA molecules with their wide range of lengths, artisans can use both denaturing agarose and polyacrylamide gels. The identity of the bands with only one ribozyme cleaved can be confirmed using antisense oligos targeting each ribozyme. The efficiency of ribozyme cleavages can be inferred from the intensities of these bands. Blotting of the non-structural region of the replicon and ofU6 snRNA (or 5S rRNA) of the host can be used to monitor the replication levels and to serve as normalization, respectively. By comparing the RNA samples extracted from transfections of miR-34a replicon and controls containing unrelated sequences, artisans can infer whether miR-34a is produced from the replicon. Further quantification of mature miR-34a may be obtained using qRT-PCR Taqman miRNA assays. Artisans can prepare small RNA libraries from these samples and perform deep sequencing using the sequencing facility of the UCLA Jonsson Comprehensive Cancer Center (Feng Guo is a member of the Cancer Center). The results from deep sequencing will not only quantitatively indicated how much miR-34a is produced but also verify—down to single-nucleotide resolution—whether the ends of miR-34a are generated precisely. Finally, the gene regulation efficacy of miR-34a produced from the replicon can be validated using immunoblotting of known miR-34a targets such as CD44.

One of the many advantages of using the Sindbis virus genome as the basis for construction of a miRNA-containing replicon is that significant levels of alphavirus replication—of full-length and subgenomic RNA—occur in a wide range of mammalian cells, and it is this replication that artisans can depend on for amplification of the miRNA. The only instance in which systematic quantification has been performed is the case of Sindbis in BHK (baby hamster kidney) cells, involving as many as 3×10⁵ copies of subgenomic RNA for each original molecule of genomic RNA. We have data indicating comparable levels of Sindbis replicon amplification in human embryonic kidney (HEK) and human pancreatic cancer (P ANC-1) cells, as assayed by emission levels from enhanced yellow fluorescent protein (EYFP) reporter gene. Also, replicons derived from a related alphavirus. Venezuelan Equine Encephalitis Virus (VEEV), developed for heterologous vaccine and protein expression (e.g. influenza hemagglutinin and green fluorescent protein (GFP)), have been shown to be strongly amplified in monkey kidney epithelial (Vero), HEK, and chicken embryo fibroblast (CEF) cells. This suggests that we can expect similarly high levels of RNA amplification of our replicons in the prostate cancer cells with which we'll be working (or, for that matter, the cancer cells ultimately chosen as targets for in vivo studies). (ii) We expect that the single-stranded small RNA produced from the Sindbis replicon can be incorporated into RISC, although probably not as efficiently as double-stranded RNAs or pre-miRNA hairpins. We believe that the design of directly expressing mature miRNAs is feasible as a large number of small RNAs can be expressed from the replicon and in this approach no complementary strand is involved to generate off-targeting effects. However, if the small RNA fails to incorporate into RISC with reasonable efficiency, we will insert into the replicon a second small RNA expression cassette to provide a complementary strand. The annealing of these RNA strands will produce a miRNA duplex identical to the natural sequence. Alternatively, we will insert a pre-miRNA sequence in place of the mature miRNA sequence in the replicon, so that DICER becomes involved in processing the miRNA for “pick-up” by the RISC machinery. (iii) If, and only if, we have time to do so, we may also try making and testing other RNAi-replicons, which take advantage in different ways of the replication-dependent (i.e., amplification of) subgenomic RNA synthesis in plus-strand viruses like Sindbis. For example, instead of flanking the miRNA sequence of interest with ribozymes (which results in loss of this sequence from most of the replicons as discussed above), we will also make a similar construct but with the reverse complement of the miRNA sequence, flanked by reverse complements of the ribozymes, so that self-cleavage is active only in the minus-strand complement. In this case the miRNA sequence in the minus strand can be deleted in most of the minus strands, and this large fraction will yield the mature miRNA of interest. i.e., the antisense sequence complementary to the mRNA.

This work is the crucial step in the development of an effective means of small RNA expression and in vivo gene repression targeting many different cancers. To transition from the current in vitro studies we will need to protect the miRNA-containing replicons and target them for uptake by specific cells. Recent work of ours has been focused on the in vitro packaging of heterologous-gene-containing replicons, i.e., on the synthesis of virus-like particles (VLPs) from purified RNA and viral capsid protein. The capsid protein of choice is that of the cowpea chlorotic mottle virus (CCMV), because of its ability to efficiently package in vitro RNA molecules of all sequences and with a wide range of lengths. Further, it has been shown that VLPs reconstituted in this way with CCMV capsid protein, when transfected into mammalian cells, make available their messenger RNA content to the ribosomal machinery. In the case of heterologous genes under the control of a subgenomic promoter, as in the case of the viral-derived replicons discussed here, we find a correspondingly high level of protein expression following VLP transfection. Finally, we have data providing evidence that in vitro reconstituted CCMV VLPs can be wrapped by liposomes, and the liposomes in turn functionalized with ligands targeting receptors over-expressed in cancer cells. The resulting enveloped virus-like particles (EVLPs) will provide the platform for in vivo delivery and targeting of self-amplifying mature miRNAs for oncogene down-regulation.

### Tables

TABLE S1
Sequence of CCS (+).
Restriction sites in typewriter font; base-paired
ribozyme stems bigger and underlined; miR-34a sequence and catalytic ribozyme
residues that base-pair with miR-34a bolded and italicized.

ATATG(5′ RE, NdeI)
GGGAGATGCCACTGATGAGTCCGTGAGGACGAAACGGTATACCGTC
(SEQ ID NO: 1) (Hammerhead ribozyme (missing 5′ pentaloop))
TGGCAGTGTCTTAGCTGGTTGT (miR-34A)
AAATATATTTGCAATCAAAAGTTATCAGGCATGCACCTGGTAGCTAGTCT
TTAAACCAATAGATTGCATCGGTTTAAAAGGCAAGACCGTCAAATTGCGG
GAAAGGGGTCAACAGCCGTTCAGTACCAAGTCTCAGGGGAAACTTTGAGA
TGGCCTTGCAAAGGGTATGGTAATAAGCTGACGGACATGGTCCTAACCAC
GCAGCCAAGTCCTAAGTCAACAGATCTTCTGTTGATATGGATGCAGTTCAC
AGACTAAATGTCGGTCGGGGAAGATGTATTCTTCTCATAAGATATAGTCG
GACCTCTCCTTAATGGGAGCTAGCGGATGAAGTGATGCAACACTGGAGCC
GCTGGGAACTAATTTGTATGCGAAAGTATATTGATTAGTTTTGGAGTACTC
(SEQ ID NO: 2) (Tetrahymena Group I intron) ACCGGT (3′ RE, Agei) (SEQ ID
NO: 3) GGGCGTACG (SEQ ID NO: 4) (Nodamura RNA1 ORF, RNA3 ORF).

TABLE S2
Table of Primers used in RT-qPCR of
NodaMiRNA full-length and microRNA molecules.

| | | | Annealing |
|---|---|---|---|
| RNA Molecule | | | Temp |
| Amplified | | Sequence (5′-3′) | (° C.) |
| NodaMiRNA Full-length | Forward | TAAGCGGCAGGCGGAAG | 60 |
| | | (SEQ ID NO: 5) | |
| | Reverse | TAGTTCGGGTTTCGTCAGATGG | 60 |
| | | (SEQ ID NO: 6) | |
| miR-34A | Forward | TGGCAGTGTCTTAGCTGGTTG | 60 |
| | | (SEQ ID NO: 7) | |
| | Reverse | GCGAGCACAGAATTAATACGA | 60 |
| | | (SEQ ID NO: 8) | |
| Adapter (miR-34A) | | GCGAGCACAGAATTAATACGAC | 65 |
| | | TCACTATAGGTTTTTTTTTTTTTTTAC | |
| | | (SEQ ID NO: 9) | |

Table S2: Table of Primers used in RT-qPCR of NodaMiRNA full-length and
microRNA molecules.

### REFERENCES

Ball L A. 1995. Requirements for the self-directed replication of flock house virus RNA 1. J Virol 69: 720-727.
Ball L A, Amann J M, Garrett B K. 1992. Replication of nodamura virus after transfection of viral RNA into mammalian cells in culture. J Virol 66: 2326-2334.
Biddlecome A, Habte H H, McGrath K M, Sambanthamoorthy S, Wurm M, Sykora M M, Knobler C M, Lorenz I C, Lasaro M, Elbers K, et al. 2019. Delivery of self-amplifying RNA vaccines in in vitro reconstituted virus-like particles. PloS One 14: e0215031.
Brito L A, Kommareddy S, Maione D, Uematsu Y, Giovani C. Berlanda Scorza F, Otten G R, Yu D. Mandl C W, Mason P W, et al. 2015. Chapter Seven—Self-Amplifying mRNA Vaccines. In Advances in Genetics (eds. L. Huang, D. Liu, and E. Wagner), Vol. 89 of Nonviral Vectors for Gene Therapy, pp. 179-233, Academic Press http://www.sciencedirect.com/science/article/pii/S0065266014000066 (Accessed Nov. 14, 2019).
Chang T-C, Wentzel E A, Kent O A, Ramachandran K, Mullendore M, Lee K H, Feldmann G, Yamakuchi M, Ferlito M, Lowenstein C J, et al. 2007. Transactivation of miR-34a by p53 broadly influences gene expression and promotes apoptosis. Mol Cell 26: 745-752.
Gitlin L, Hagai T, LaBarbera A, Solovey M, Andino R. 2014. Rapid Evolution of Virus Sequences in Intrinsically Disordered Protein Regions. PLoS Pathog 10. https://www.ncbi.nlm.nih.gov/pmc/articles/PMC4263755/(Accessed Nov. 24, 2019).
Gold L, Janjic N, Jarvis T, Schneider D, Walker J J, Wilcox S K, Zichi D. 2012. Aptamers and the RNA World, Past and Present. Cold Spring Harb Perspect Biol 4: a003582.
Griffiths-Jones S. 2010. miRBase: microRNA sequences and annotation. Curr Protoc Bioinforma Chapter 12: Unit 12.9.1-10.
Guo F, Cech T R. 2002. In vivo selection of better self-splicing introns in Escherichia coli: The role of the P1 extension helix of the Tetrahymena intron. RNA 8: 647-658.
Guo Y, Liu J, Elfenbein S J, Ma Y, Zhong M, Qiu C, Ding Y, Lu J. 2015. Characterization of the mammalian miRNA turnover landscape. Nucleic Acids Res 43: 2326-2341.
Hayes J, Peruzzi P P, Lawler S. 2014. MicroRNAs in cancer: biomarkers, functions and therapy. Trends Mol Med 20: 460-469.
Johnson K L, Ball L A. 1999. Induction and Maintenance of Autonomous Flock House Virus RNA1 Replication. J Virol 73: 7933-7942.
Johnson K L, Price B D, Eckerle L D, Ball L A. 2004. Nodamura Virus Nonstructural Protein B2 Can Enhance Viral RNA Accumulation in both Mammalian and Insect Cells. J Virol 78: 6698-6704.
Khromykh A A. 2000. Replicon-based vectors of positive strand RNA viruses. Curr Opin Mol Ther 2: 555-569.
Leonhardt C. Schwake G, Stögbauer T R, Rappl S, Kuhr J-T, Ligon T S, Radler J O. 2014. Single-cell mRNA transfection studies: delivery, kinetics and statistics by numbers. Nanomedicine Nanotechnol Biol Med 10: 679-688.
Li N, Fu H, Tie Y, Hu Z, Kong W, Wu Y, Zheng X. 2009. miR-34a inhibits migration and invasion by down-regulation of c-Met expression in human hepatocellular carcinoma cells. Cancer Lett 275: 44-53.
Lim L P, Glasner M E, Yekta S, Burge C B, Bartel D P. 2003. Vertebrate microRNA genes. Science 299:1540.
Lindenbach B D, Sgro J-Y, Ahlquist P. 2002. Long-distance base pairing in flock house virus RNA1 regulates subgenomic RNA3 synthesis and RNA2 replication. J Virol 76: 3905-3919.
Liu C, Kelnar K, Liu B, Chen X, Calhoun-Davis T, Li H, Patrawala L, Yan H, Jeter C, Honorio S, et al. 2011. The microRNA miR-34a inhibits prostate cancer stem cells and metastasis by directly repressing CD44. Nat Med 17: 211-215.
Lundstrom K. 2016. Replicon RNA Viral Vectors as Vaccines. Vaccines 4. https://www.ncbi.nlm.nih.gov/pmc/articles/PMC5192359/(Accessed Nov. 14, 2019).
Maharaj P D, Mallajosyula J K, Lee G, Thi P, Zhou Y, Kearney C M, McCormick A A. 2014. Nanoparticle encapsidation of Flock house virus by auto assembly of Tobacco mosaic virus coat protein. Int J Mol Sci 15: 18540-18556.
Mansoori B, Sandoghchian Shotorbani S, Baradaran B. 2014. RNA Interference and its Role in Cancer Therapy. Adv Pharm Bull 4: 313-321.
Martinez J, Patkaniowska A, Urlaub H, Lührmann R, Tuschl T. 2002. Single-stranded antisense siRNAs guide target RNA cleavage in RNAi. Cell 110: 563-574.
Miller D J, Ahlquist P. 2002. Flock house virus RNA polymerase is a transmembrane protein with amino-terminal sequences sufficient for mitochondrial localization and membrane insertion. J Virol 76: 9856-9867.
Navarro F, Lieberman J. 2015. miR-34 and p53: New Insights into a Complex Functional Relationship. PLOS ONE 10: e0132767.
Nimjee S M, Rusconi C P, Sullenger B A. 2005. Aptamers: An Emerging Class of Therapeutics. Annu Rev Med 56: 555-583.
Paudel B P, Rueda D. 2014. Molecular crowding accelerates ribozyme docking and catalysis. J Am Chem Soc 136: 16700-16703.
Price S R, Ito N, Oubridge C. Avis J M. Nagai K. 1995. Crystallization of RNA-protein complexes I. Methods for the large-scale preparation of RNA suitable for crystallographic studies. J Mol Biol 249: 398-408.
Shi R, Chiang V L. 2005. Facile means for quantifying microRNA expression by real-time PCR. BioTechniques 39: 519-525.
Tros de Ilarduya C. Sun Y, Düzgünş N. 2010. Gene delivery by lipoplexes and polyplexes. Eur J Pharm Sci Off J Eur Fed Pharm Sci 40: 159-170.
Venter P A, Schneemann A. 2008. Recent insights into the biology and biomedical applications of Flock House virus. Cell Mol Life Sci CMLS 65: 2675-2687.
Yamakuchi M, Ferlito M, Lowenstein C J. 2008. miR-34a repression of SIRT1 regulates apoptosis. Proc Natl Acad Sci USA 105: 13421-13426.
Zuker M. 2003. Mfold web server for nucleic acid folding and hybridization prediction. Nucleic Acids Res 31: 3406-3415.
Conversion: weight—moles (for nucleic acids)|Practical Molecular Biology. http://molbiol.edu.ru/eng/scripts/01_07.html (Accessed Nov. 22, 2019a).
Useful Numbers for Cell Culture—US. https://www.thermofisher.com/us/en/home/references/gibco-cell-culture-basics/cell-culture-protocols/cell-culture-useful-numbers.html (Accessed Nov. 22, 2019c).

All publications mentioned herein (e.g., Torres-Salgado; Eury Phys J E Soft Matter. 2023 Dec. 5; 46(12):118; and the references listed above) are incorporated herein by reference to disclose and describe aspects, methods and/or materials in connection with the cited publications.

1. A composition of matter comprising a polynucleotide, wherein the polynucleotide comprises:
(a) a polynucleotide sequence encoding a RNA-dependent-RNA-polymerase;
(b) a T2A self-cleaving peptide sequence;
(c) a polynucleotide sequence comprising a first ribozyme;
(d) a polynucleotide sequence comprising a cargo polynucleotide; and
(e) a polynucleotide sequence comprising a second ribozyme; wherein (a)-(e) are disposed in the polynucleotide such that when the polynucleotide is transduced into a mammalian cell:
the RNA dependent RNA polymerase encoded by the polynucleotide is made;
the polynucleotide is replicated; and
the first and second ribozymes cleave the polynucleotide such that a polynucleotide fragment consisting of the cargo polynucleotide is generated.

2. The composition of claim 1, wherein the cargo polynucleotide comprises a MiRNA.

3. The composition of claim 1, wherein the polynucleotide is disposed within a vector.

4. The composition of claim 1, further comprising at least one pharmaceutically acceptable excipient.

5. The composition of claim 1, further comprising a lipid selected to form liposomes.

6. A method of making a composition of claim 1, the method comprising:
forming a polynucleotide, wherein the polynucleotide comprises:
(a) a polynucleotide sequence encoding a RNA dependent RNA polymerase;
(b) a T2A self-cleaving peptide sequence;
(c) a polynucleotide sequence comprising a first ribozyme;
(d) a polynucleotide sequence comprising a cargo polynucleotide; and
(e) a polynucleotide sequence comprising a second ribozyme; and
organizing (a)-(e) in the polynucleotide such that when the polynucleotide is transduced into a mammalian cell:
the RNA-dependent-RNA-polymerase encoded by the polynucleotide is made;
the polynucleotide is replicated; and
the first and second ribozymes cleave the polynucleotide such that a polynucleotide fragment consisting of the cargo polynucleotide is generated;
so that the composition is made.

7. The method of claim 6, wherein:
the polynucleotide sequence encoding a RNA-dependent-RNA-polymerase encodes a Nodaviridae RNA-dependent-RNA-polymerase;
the polynucleotide sequence comprising the first ribozyme or the polynucleotide sequence comprising the second ribozyme comprise a Hammerhead ribozyme and a Tetrahymena ribozyme; and/or
the polynucleotide sequence comprising a cargo polynucleotide consists of a miRNA.

8. The method of claim 6, wherein (a)-(e) in the polynucleotide are organized such that when the polynucleotide is transduced into a mammalian BHK-21 cell, at least 5,000 polynucleotide fragments consisting of the cargo polynucleotide are generated

9. The method of claim 6, wherein the polynucleotide is formed in vitro.

10. The method of claim 6, wherein the polynucleotide is formed in vivo.

11. A method of delivering a cargo polynucleotide into a mammalian cell, the method comprising combining the mammalian cell with a composition of claim 1 under conditions selected so that the cargo polynucleotide is transduced into the mammalian cell.

12. The method of claim 11, wherein the mammalian cell is a cancer cell.

13. The method of claim 11, wherein the method delivers a cargo polynucleotide into a mammalian cell in vivo.

14. The method of claim 11, wherein when the cargo polynucleotide is transduced into the mammalian cell, at least 2,000 polynucleotide fragments consisting of the cargo polynucleotide are generated.

15. The method of claim 11, wherein the cargo polynucleotide is transduced into the mammalian cell via lipofection.

16. A composition of matter comprising a polynucleotide, wherein the polynucleotide comprises:
(a) a polynucleotide sequence encoding a RNA-dependent-RNA-polymerase;
(b) a T2A self-cleaving peptide sequence;
(c) a polynucleotide sequence comprising a first ribozyme;
(d) a cargo polynucleotide;
(e) a lipid selected to form liposomes; and
(e) a polynucleotide sequence comprising a second ribozyme; wherein (a)-(e) are disposed in the polynucleotide such that when the polynucleotide is transduced into a mammalian cell:
the RNA dependent RNA polymerase encoded by the polynucleotide is made;
the polynucleotide is replicated; and
the first and second ribozymes cleave the cargo polynucleotide such that a polynucleotide fragment is generated.

17. The composition of claim 16, wherein the cargo polynucleotide comprises a MiRNA.

18. The composition of claim 16, wherein the cargo polynucleotide encodes a polypeptide.

19. The composition of claim 16, wherein the cargo polynucleotide is disposed within a vector.

20. The composition of claim 16, further comprising at least one pharmaceutically acceptable excipient.
